SEQUENCE LISTING (3 sequences)

SEQ ID NO: 1 - RNA, 19 nt, Artificial Sequence
  Epac1siRNA antisense sequence
aucacuguauaccgguucc

SEQ ID NO: 2 - RNA, 19 nt, Artificial Sequence
  Syndecan-2 siRNA antisense sequence
cucuggacucucuacaucc

SEQ ID NO: 3 - RNA, 19 nt, Artificial Sequence
  NDST - 1 siRNA antisense sequence
uuuauuagcaguuaguucg

(19) United States
(12) Patent Application Publication
(10) Pub. No.: US 2011/0060029 A1
(43) Pub. Date: Mar. 10, 2011

(54) METHOD OF TREATING CANCER BY MODULATING EPAC

(76) Inventors: Kosaku Iwatsubo, Fort Lee, NJ (US); Erdene Balijinnyam, Bloomfield, NJ (US); Ishikawa Yoshihiro, Newark, NJ (US)

(21) Appl. No.: 12/756,846

(22) Filed: Apr. 8, 2010

Related U.S. Application Data
(60) Provisional application No. 61/212,274, filed on Apr. 8, 2009.

(51) Int. Cl. A61K 31/7105; A61K 31/7088; A61P 35/00

(52) U.S. Cl. 514/44 A

(57) ABSTRACT

Methods of treating cancer by preventing, mitigating, and/or inhibiting cancer metastasis in tumors expressing Epac by inhibiting the activity of exchange proteins directly activated by cyclic AMP (Epac) or one or more proteins within the Epac-induced carcinoma migration pathway.

### CROSS REFERENCE TO RELATED APPLICATIONS

The instant application claims 35 U.S.C. §119(e) priority to U.S. Provisional Patent Application Ser. No. 61/212,274 filed Apr. 8, 2009, the disclosure of which is incorporated by reference herein.

### FIELD OF THE INVENTION

The present invention relates to methods of treating cancer by targeting the pathway of activity associated with exchange proteins directly activated by cyclic AMP (Epac) and inhibiting metastasis.

### BACKGROUND OF THE INVENTION

Melanoma is one of the most malignant forms of human skin cancer and has poor prognosis due to its strong metastic ability. It is a major cancer worldwide, with the median life span of advanced stage patients being less than a year due, in part, to little or no effective therapies available after metastasis to vital organs. Metastasis of melanoma, and generally for tumor cell migration, is conventionally understood as the migration of individual cells that detach from the primary tumor, enter lymphatic vessels or the bloodstream, and seed in distant organs. Despite numerous efforts in the research field, understanding and controlling melanoma migration/metastasis have been unsuccessful.

Molecular events associated with tumor cell migration are influenced, in part, by interactions between the cancer cells and their extra-cellular matrix (ECM) components. Heparan sulfate (HS), for example, is a major ECM component and is known to contribute to cell motility of metastic cancers by binding to and regulating key signaling molecules. While its regulation is poorly understood, HS is known to control cellular migration through an interaction with the protein syndecan-2, a member of cell surface heparan sulfate proteoglycans (HSPGs). Previous studies have shown that HS/syndecan interaction facilitates syndecan-translocation and clustering into rafts and is correlative with microtube polymerization. These rafts provide the lipid-rich microdomains in the plasma membrane that serve as a platform for the binding of syndecans to the ECM and facilitating cell mobility.

Recently, the exchange protein directly activated by cAMP (Epac) also was suggested to be involved in cell migration regulation. Epac, much like the signaling pathway of protein kinase A (PKA), is a cAMP-regulated guanine nucleotide exchange factor that mediates signal transduction properties of the second messenger cAMP. Two isoforms of Epac are known to exist, Epac1 and Epac2, both of which are activated in living cells by physiologically relevant concentrations of cAMP. Once activated, Epac stimulates GTP to GDP exchange and activates one or multiple small-molecular-weight G proteins, such as the Rap or Rac superfamily of proteins. This activity has been shown to contribute to numerous downstream cellular functions, including, inter alia, secretion, Ca²⁺ signaling, proliferation and apoptosis. While recent reports also indicate involvement of Rap1-Epac interactions in regulating cell migration, it was previously unknown whether such molecular mechanisms contributed to or otherwise increased metastasis of malignant carcinomas, such as melanoma.

Accordingly, there is a need in the art for the further characterization of the Epac function in cell motility, particularly with respect to carcinoma metastasis. There also remains a need for greater understanding of the role of Epac in the progress of melanoma and of its evaluation as a potential target site for a cancer therapeutic. The instant invention addresses these needs.

### SUMMARY OF THE INVENTION

The present invention relates to methods of treating cancer by preventing, mitigating, and/or inhibiting cancer metastasis. More specifically, the instant invention relates to the discovery that Epac proteins are key regulating mechanisms for metastasis and cell migration. A novel pathway is thereby presented herein for the development of anticancer therapeutics that prevent, mitigate and/or treat carcinoma migration and cancer development.

In one aspect, the instant invention relates to a method for inhibiting, mitigating or preventing cancer metastasis in a subject by administering a therapeutically effective amount of one or more Epac inhibitors. Epac inhibitors may be specifically adapted to inhibit the expression or activation of an Epac protein, such as but not limited to Epac 1 or Epac 2. These inhibitors may include a compound, or a pharmaceutically acceptable salt thereof, or a biological agent. Biological agents may include a nucleic acid (e.g., DNA enzyme, an antisense RNA, an siRNA, a shRNA, and an aptamer) or any other agent discussed herein. In one embodiment, the nucleic acid is an siRNA having an antisense sequence of SEQ ID NO: 1.

In another aspect, the instant invention relates to a method for inhibiting, mitigating or preventing cancer metastasis in a subject by administering a therapeutically effective amount of one or more inhibitors to a subject wherein the inhibitors target one or more proteins within an Epac-induced carcinoma migration pathway. Such a pathway may include, but is not limited to, the expression or activation of syndecan-2, NDST-1, or Rap-1. Inhibitors may include a compound, or a pharmaceutically acceptable salt thereof, or a biological agent. Biological agents may include a nucleic acid (e.g., DNA enzyme, an antisense RNA, an siRNA, a shRNA, and an aptamer) or any other agent discussed herein. In one embodiment, the nucleic acid is an siRNA inhibiting expression of syndecan-2 and having an antisense sequence of SEQ ID NO: 2. In another embodiment, the nucleic acid is an siRNA inhibiting expression of NDST-1 and having an antisense sequence of SEQ ID NO: 3.

### BRIEF DESCRIPTION OF THE FIGURES

FIG. 1 illustrates that Epac1 increases melanoma cell migration.

FIG. 2 illustrates that Epac1 activates syndecan-2 translocation into rafts in SK-Mel-2.

FIG. 3 illustrates that Epac1 increases syndecan-2 translocation via tubulin polymerization in SK-Mel-2.

FIG. 4 illustrates that Epac regulates tubulin polymerization via phosphoinositol-3 kinase (PI3K) in SK-Mel-2.

FIG. 5 illustrates that Epac increases cell migration by Heparan sulfate (HS) production in SK-Mel-2.

FIG. 6 illustrates that Epac increases lung metastasis of Cloudman S91 melanoma in mice.

### DETAILED DESCRIPTION OF THE INVENTION

### Definitions

As used herein, the term “biological agent” or “biological agents” include any agent known in the art such as, but not limited to, proteins or protein-based molecule, such as a mutant ligand, antibody, or the like, and nucleic acids or nucleic acid-based entities and the vectors used for their delivery.

As used herein, the term “compound” or “compounds” refers to conventional chemical compounds (e.g., small organic or inorganic molecules). To this end, the terms small molecule and compounds are interchangeable.

As used herein, “effective amount” is an amount sufficient to effect beneficial or desired clinical or biochemical results. An effective amount can be administered one or more times. For purposes of this invention, an effective amount of an inhibitor compound is an amount that is sufficient to palliate, ameliorate, stabilize, reverse, slow or delay the progression of the disease state.

As used herein, the term “Epac inhibitor” refers to a compound or any biological agent that decreases the activity of Epac in a cell and decreases cancer cell or carcinoma migration by any measurable amount, as compared to such a cell in the absence of such an inhibitor. Epac may include, but is not limited to Epac1 or Epac2.

As used herein, the term “Epac-induced carcinoma migration pathway inhibitor” refers to a compound or any biological agent that decreases cancer cell or carcinoma migration in a cell by targeting proteins or molecules associated with Epac-induced migration, as compared to a cell in the absence of such an inhibitor. Such proteins or molecules include, but are not limited to, N-deacetylase/N-sulfotranferase-1 (NDST-1), syndecan-2 or Rap-1.

A composition is said to be “pharmacologically or physiologically acceptable” if its administration can be tolerated by a recipient animal and is otherwise suitable for administration to that animal. Such an agent is said to be administered in a “therapeutically effective amount” if the amount administered is physiologically significant. An agent is physiologically significant if its presence results in a detectable change in the physiology of a recipient patient.

As used herein, with respect to administering an inhibitor, the terms “mitigate” or “mitigating” refers to reducing the progression, e.g., metastasis, of a cancer. It may include executing a protocol, which may include administering one or more drugs to a patient (human or otherwise), in an effort to reduce signs or symptoms of the disease.

As used herein, the terms “metastasis,” “cancer migration,” or “carcinoma migration” means the spread of tumor cells from the site of origin to other areas through lymphatic or blood vessels. In a broad sense, metastasis also means the direct extension of tumor cells through serous body cavity or other space.

As used herein, with respect to administering an inhibitor, the terms “prevent,” or “preventing” refers to prophylactic treatment for halting a disease or condition. It may include executing a protocol, which may include administering one or more drugs to a patient (human or otherwise), in an effort to prevent signs or symptoms of the disease. In certain embodiments, prophylactic treatment prevents worsening of a disease or condition.

As used herein, the terms “siRNA molecule,” “shRNA molecule,” “RNA molecule,” “DNA molecule,” “cDNA molecule” and “nucleic acid molecule” are each intended to cover a single molecule, a plurality of molecules of a single species, and a plurality of molecules of different species.

As used herein, the term “subject” refers to any animal (e.g., a mammal), including, but not limited to humans, non-human primates, rodents, and any other animal, which is to be the recipient of a particular treatment. Typically, the terms “subject” and “patient” are used interchangeably herein in reference to a human subject.

As used herein, with respect to administering an inhibitor, the terms “treat,” “treating,” or “treatment” refers to therapeutic treatment for halting or reducing a disease or condition. It may include executing a protocol, which may include administering one or more drugs to a patient (human or otherwise), in an effort to alleviate signs or symptoms of the disease. In certain embodiments, therapeutic treatment prevents worsening of a disease or condition.

The present invention relates to methods of treating cancer by preventing, mitigating, and/or inhibiting cancer metastasis. More specifically, the instant invention relates to the discovery that Epac proteins are key regulating mechanisms for carcinoma metastasis and cell migration. A novel pathway is thereby presented herein for the development of anticancer therapeutics that prevent, mitigate and/or treat such migration and cancer development. As illustrated below, it was surprisingly discovered that Epac regulates both HS production and syndecan-2 translocation/raft formation in cancer cell metastasis both in vitro and in vivo. Epac activation, either by specific agonist or overexpression, is shown herein to alter HS signaling at multiple levels, most notably, by regulating HS biosynthesis. This results in cell interaction with HS and enhances translocation and raft formation of HS-bonding protein syndecan-2. Thus, Epac activation is demonstrated to be a major determinant in regulating multiple aspects of carcinoma metastasis.

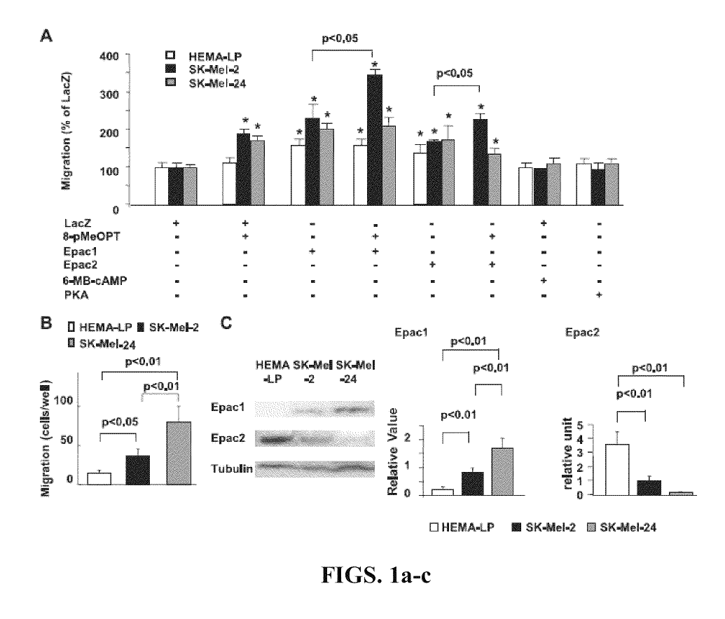

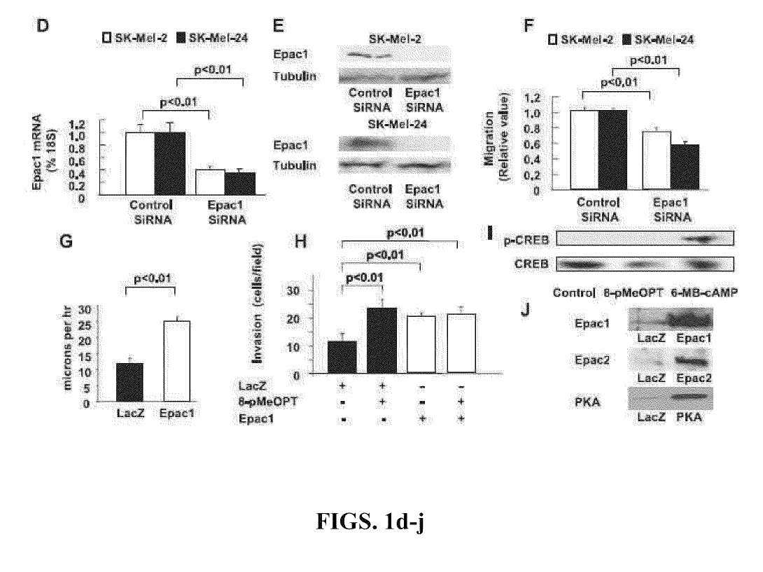

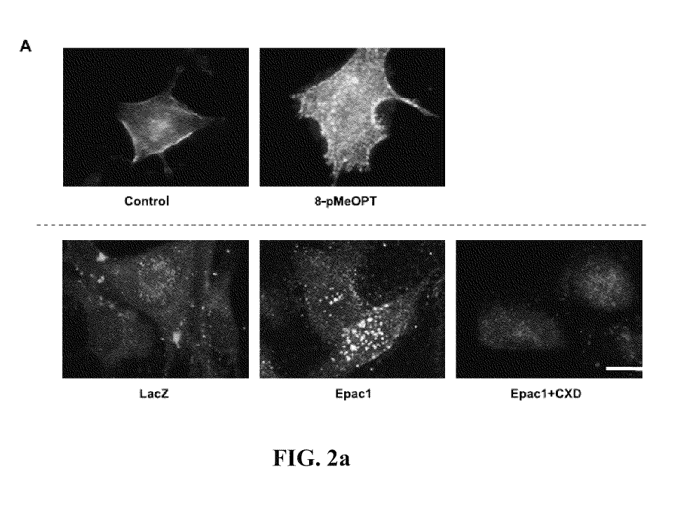

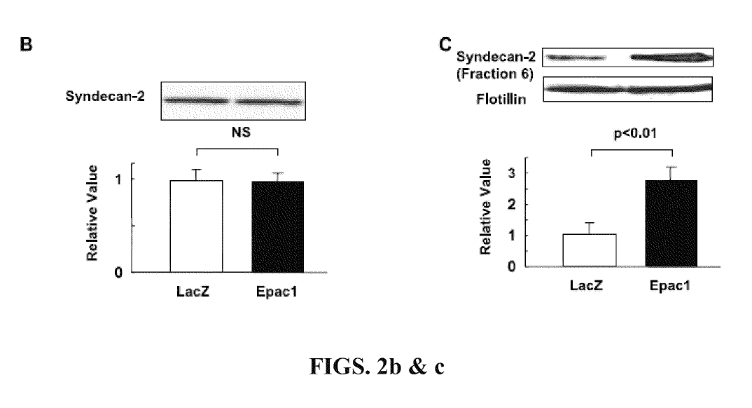

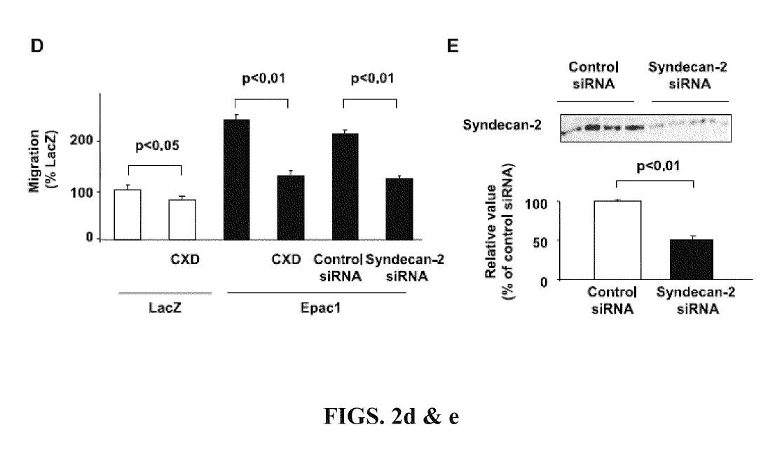

The studies discussed herein confirm that the Epac pathway is, at least in part, responsible for carcinomal cell migration. Referring to FIGS. 1 and 2, Epac1 and Epac2 activation in carcinoma cells is illustrated as exhibiting an overall increase in migration, as compared to the lack of migration in non-carcinogenic Epac expressing cell lines. Such an effect was confirmed when these cells were treated with an Epac inhibiting agent, in this case siRNA, and resulted in decreased Epac expression levels, as well as a decreased cellular migration.

Epac activation was further observed to increase HS production and the formation of metastatic nodules. While not intending to be bound by theory, it is believed that increased expression of Epac results in increased translation of N-deacetylase/N-sulfotranferase-1 (NDST-1), a known rate-limiting enzyme for HS biosynthesis. Indeed, when NDST-1 was downregulated by siRNA, Epac-induced HS production and carcinoma migration were significantly decreased.

In concert with increased HS production, Epac activated cells also exhibited an overall increase of the glycanated form of syndecan-2, i.e., the HS bound syndecan-2. More specifically, immunocytochemical studies provided below showed that Epac activation or overexpression enhanced colocalization of syndecan-2 with lipid rafts, and that this colocalization could be inhibited by a raft-disrupting agent. Indeed, the administration of siRNA targeting syndecan-2 resulted in an overall decrease of Epac-induced carcinoma migration. This suggests that Epac overexpression in motile carcinomas results in an overall increase of syndecan-2 granulation and raft formation, further supporting the notion that Epac-induced pathway enhances cell metastasis.

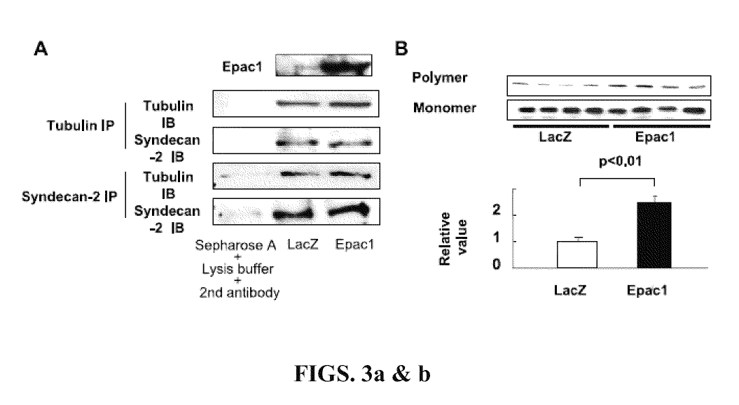

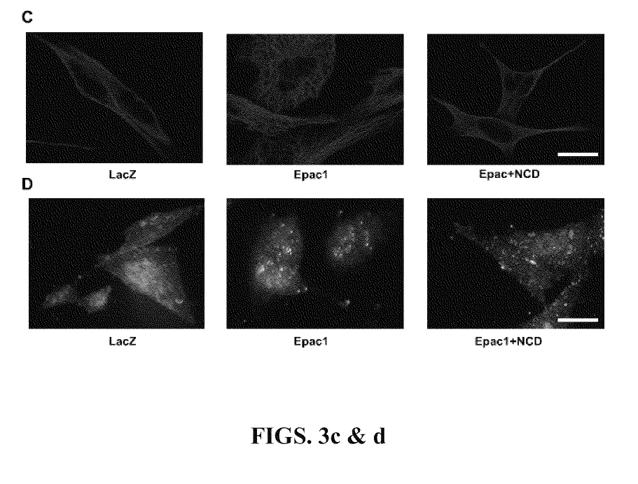

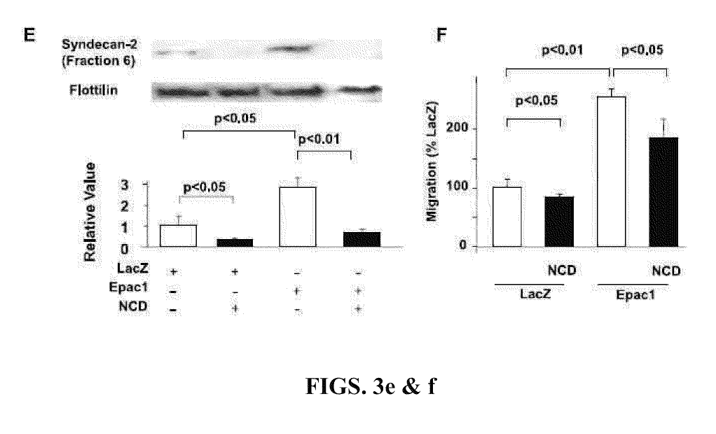

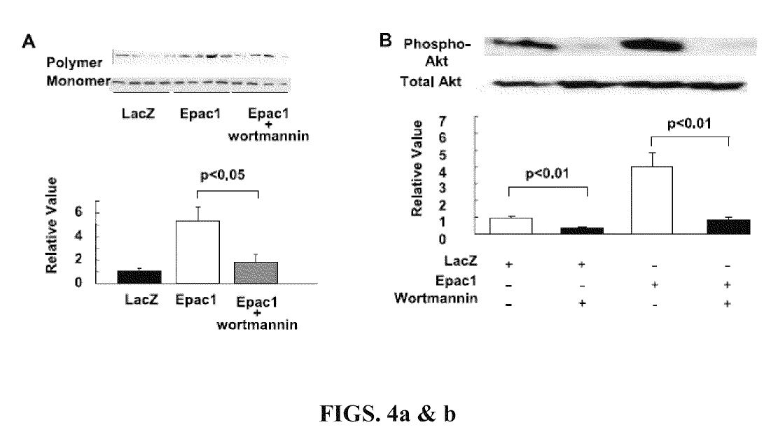

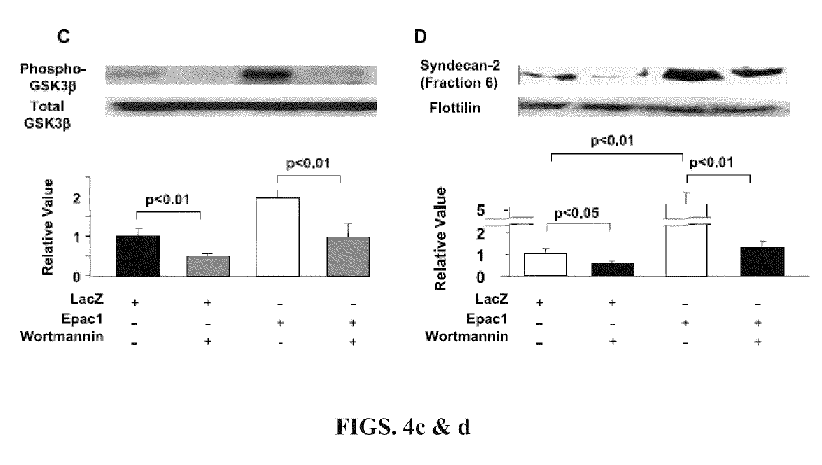

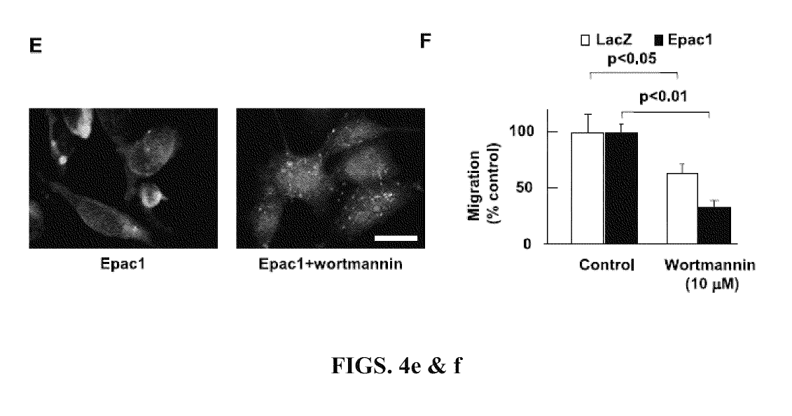

Syndecan-2 translocation was also shown to be mediated by tubulin polymerization via phosphoinositol-3 kinsase (PI3K). P13K regulates tubulin via the Akt/GSK3β pathway. Epac is known to phosphorylate and inactivate GSK3β, which results in increased tubulin polymerization. P13K is also activated by Epac, thus, implicating that Epac is an upstream mediator of the PI3K/Akt/GSK3β pathway. This implication was confirmed in FIGS. 3 and 4, which demonstrated that Epac increases tubulin polymerization via activation of the P13K/Akt/GSK3β pathway.

Based on the foregoing, the instant invention relates to the administration of one or more inhibitors for the purpose of preventing, mitigating, or inhibiting cancer metastasis. This invention, thereby, can be used to effectively and specifically mitigate, prevent or inhibit pathological conditions related to cancer, particularly malignant carcinomas such as melanoma. By administering one or a combination of Epac inhibitors or inhibitors of the pathway associated Epac-induced metastasis, one would effectively reduce the incidence of cancer motility, thereby, drastically improving the patient's prognosis.

Inhibitors may be used for the treatment, mitigation, and/or prevention of any metastatic cancer including without limitation carcinoma, melanoma and sarcoma. Subtypes of cancer may include without limitation bladder carcinoma, brain tumor, breast cancer, cervical cancer, colorectal cancer, esophageal cancer, endometrial cancer, hepatocellular carcinoma, gastrointestinal stromal tumor (GIST), laryngeal cancer, lung cancer, osteosarcoma, ovarian cancer, pancreatic cancer, prostate cancer, renal cell carcinoma, skin cancer, or thyroid cancer. In particular, the cancer may be melanoma, particularly metastic melanoma. Again, the instant invention is not so limiting and may include any form or cancer, particularly a form that uses a pathway associated with Epac for achieving cellular motility.

In one embodiment, the inhibitor may be a compound that targets Epac or the pathway associated with Epac-induced metastasis (e.g., enzymes, proteins, mRNA expression, etc). In one embodiment the compound is Brefeldin A, a small hydrophobic compound produced by toxic fungi that binds at the Rap-GDP/Epac interface, as set forth in US20090169540, the contents of which are incorporated herein by reference. In another embodiment, compounds may also include the class of Epac inhibitory compounds disclosed within US20070197482, the contents of which are incorporated by reference herein. One of ordinary skill in the art would appreciate that chemical analogs of one or more of the foregoing compounds would achieve similar results. The instant invention is not necessarily limited to compounds targeting Epac directly, however. In further embodiments, such compounds may target downstream mechanisms of the Epac-induced metastasis pathway. Such inhibitors may include, but are not limited to, syndecan-2 inhibitors, NDST-1 inhibitors, Rap-1 inhibitors, or inhibitors of other enzymes discussed herein or associated with such a pathway.

In addition to the use of compounds described above, inhibitors may also include other molecular or biologic agents. In one embodiment, the inhibitor is a nucleic acid molecule capable of inhibiting the expression of Epac or one or more proteins within the Epac-induced carcinoma migration pathway. Such nucleic acids may include, but are not limited to a nucleic acid encoding an antisense RNA, an siRNA, a shRNA, dsRNA, DNA enzyme, or aptamer, and can be designed based on criteria well known in the art or otherwise discussed herein.

siRNAs (short interfering RNAs) are double-stranded RNA (dsRNA) molecules that induce the sequence-specific silencing of genes by the process of RNA interference (RNAi) in multiple organisms, including humans. An siRNA typically targets a 19-23 base nucleotide sequence in a target mRNA. Naturally occurring siRNAs tend to be 21-28 nucleotides in length and occur naturally in cells. However, synthetic siRNAs have been used to specifically target gene silencing in mammalian cells. Alternative aspects of siRNA technology include chemical modifications that increase the stability and specificity of the siRNAs, and a variety of delivery methods and in vivo model systems. siRNA sequences can for example be designed using software algorithms that are commercially available. For example, the algorithm BLOCK-iT™ RNAi Designer (Invitrogen, Calif.), can be used to select appropriate sequences for an siRNA directed against Epac or one or more proteins within the Epac-induced migration pathway.

In one embodiment, for example, the Epac inhibitor includes an siRNA antisense sequence which includes, but is not limited to, 5′-AUCACUGUAUACCGGUUCC-3′ (SEQ ID NO: 1). In a further embodiment, the inhibitor is an siRNA antisense inhibitor of syndecan-2 having an antisense sequence which includes, but is not limited to, 5′-CUCUGGACUCUCUACAUCC-3′ (SEQ ID NO: 2). In an even further embodiment, the inhibitor is an siRNA antisense inhibitor of NDST-1, having an antisense sequence which includes, but is not limited to, 5′-UUUAUUAGCAGUUAGUUCG-3′ (SEQ ID NO: 3). Such antisense RNA molecules would similarly contain a sequence that is complementary to the RNA transcript of an the corresponding gene, and which can bind to the transcript, thereby reducing or preventing its expression in vivo. The antisense RNA molecule will have a sufficient degree of complementarity to the target mRNA to avoid non-specific binding of the antisense molecule to non-target sequences under conditions in which specific binding is desired, such as under physiological conditions. Again, the instant invention is not limited to the foregoing targets, and additional or alternative targets may include one or more of the enzymes identified or discussed herein.

Small hairpin RNA (shRNA) are also contemplated for RNAi of Epac expression or expression of one or more genes associated with the Epac-induced migration pathway. shRNA is a sequence of RNA that makes a tight hairpin turn that can be used to silence gene expression via RNA interference. These hairpin structures, once processed by the cell, are equivalent to siRNA molecules and are used by the cell to mediate RNAi of the desired protein. The use of shRNA has an advantage over siRNA transfection as the former can lead to stable, long-term inhibition of protein expression. Such shRNA may be designed using standard methodologies known in the art.

DNA enzymes may be comprised of magnesium-dependent catalytic nucleic acids of DNA that can selectively bind to an RNA substrate, such as an Epac, syndecan-2, Rap-1, or NDST-1 RNA substrate, by Watson-Crick base-pairing and potentially cleave a phosphodiester bond of the backbone of the RNA substrate at any purine-pyrimidine junction. As understood in the art, DNA enzymes are comprised of two distinct functional domains: a 15-nucleotide catalytic core that carries out phosphodiester bond cleavage, and two hybridization arms flanking the catalytic core; the sequence identity of the arms can be tailored to achieve complementary base-pairing with target RNA substrates. In the instant invention, a DNA enzyme may be used that has complementary regions that can anneal with regions on the transcript of the Epac, syndecan-2, Rap-1 or NDST-1 gene, or other molecular machinery discussed herein, such that the catalytic core of the DNA enzyme is able to cleave the transcript and prevent translation.

The inhibiting nucleic acids of the instant invention can be introduced into cells in vitro or ex vivo using techniques well-known in the art, including electroporation, calcium phosphate co-precipitation, microinjection, lipofection, polyfection, and conjugation to cell penetrating peptides (CPPs). In one embodiment, such nucleic acid can be introduced into cells in vivo by endogenous production from an expression vector(s) encoding the appropriate sequences. Such expression vectors may be comprised of any expression vectors known in the art that is operably linked to a genetic control element capable of directing expression of the nucleic acid within a cell. Expression vectors can be transfected into cells using methods generally known to the skilled artisan.

Biological agents as inhibitors are not necessarily limited to nucleic acids, however, and also may be comprised of any other agents otherwise known in the art that may be contemplated for inhibiting the expression or activation of Epac, or one or more genes/proteins associated with the Epac-induced migration pathway. Such agents may include, but are not limited to antibodies, ribozymes, proteins, or other biological agents known in the art for such purposes.

As provided herein, the clinical therapeutic indications envisioned for administration of an effective amount of one or more of the inhibitors herein include, but are not limited to, any preventative, mitigating and/or treatment regiment targeting, generally, the pathological conditions relating cancer metastasis and treatment of cancer.

Inhibitors of the present invention may be synthesized using methods known in the art or as otherwise specified herein. Unless otherwise specified, a reference to a particular compound of the present invention includes all isomeric forms of the compound, to include all diastereomers, tautomers, enantiomers, racemic and/or other mixtures thereof. Unless otherwise specified, a reference to a particular compound also includes ionic, salt, solvate (e.g., hydrate), protected forms, and prodrugs thereof. To this end, it may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19, the contents of which are incorporated by reference herein. Reference to a nucleic acid or biological agent similarly refers to the specific sequences herein or otherwise known, as well as homologues thereof.

Based on the foregoing, one or more inhibitors of Epac or pathway associated with Epac-induced migration, either alone or in combination, may be synthesized and administered as a pharmacologically acceptable therapeutic composition. The compositions of the present invention can be presented for administration to humans and other animals in unit dosage forms, such as tablets, capsules, pills, powders, granules, sterile parenteral solutions or suspensions, oral solutions or suspensions, oil in water and water in oil emulsions containing suitable quantities of the compound, suppositories and in fluid suspensions or solutions. To this end, the pharmaceutical compositions may be formulated to suit a selected route of administration, and may contain ingredients specific to the route of administration. Routes of administration of such pharmaceutical compositions are usually split into five general groups: inhaled, oral, transdermal, parenteral and suppository. In one embodiment, the pharmaceutical compositions of the present invention may be suited for parenteral administration by way of injection such as intravenous, intradermal, intramuscular, intrathecal, or subcutaneous injection. Alternatively, the composition of the present invention may be formulated for oral administration as provided herein or otherwise known in the art.

For oral administration, either solid or fluid unit dosage forms can be prepared. For preparing solid compositions such as tablets, the compound can be mixed with conventional ingredients such as talc, magnesium stearate, dicalcium phosphate, magnesium aluminum silicate, calcium sulfate, starch, lactose, acacia, methylcellulose and functionally similar materials as pharmaceutical diluents or carriers. Capsules are prepared by mixing the compound with an inert pharmaceutical diluent and filling the mixture into a hard gelatin capsule of appropriate size. Soft gelatin capsules are prepared by machine encapsulation of a slurry of the compound with an acceptable vegetable oil, light liquid petrolatum or other inert oil.

Fluid unit dosage forms or oral administration such as syrups, elixirs, and suspensions can be prepared. The forms can be dissolved in an aqueous vehicle together with sugar or another sweetener, aromatic flavoring agents and preservatives to form a syrup. Suspensions can be prepared with an aqueous vehicle with the aid of a suspending agent such as acacia, tragacanth, methylcellulose and the like.

For parenteral administration fluid unit dosage forms can be prepared utilizing the compound and a sterile vehicle. In preparing solutions the compound can be dissolved in water for injection and filter sterilized before filling into a suitable vial or ampoule and sealing. Adjuvants such as a local anesthetic, preservative and buffering agents can be dissolved in the vehicle. The composition can be frozen after filling into a vial and the water removed under vacuum. The lyophilized powder can then be scaled in the vial and reconstituted prior to use.

Dose and duration of therapy will depend on a variety of factors, including: (1) the patient's age, body weight, and organ function (liver and kidney function); (2) the nature and extent of the disease process to be treated, as well as any existing significant co-morbidity and concomitant medications being taken; and (3) drug-related parameters such as the route of administration, the frequency and duration of dosing necessary to effect a cure, and the therapeutic index of the drug. In general, the dose will be chosen to achieve serum levels of 1 ng/ml to 100 ng/ml with the goal of attaining effective concentrations at the target site of approximately 1 μg/ml to 10 μg/ml. Using factors such as this, a therapeutically effective amount may be administered so as to ameliorate the targeted symptoms of and/or treat or prevent obesity or diseases related thereto. Determination of a therapeutically effective amount is well within the capabilities of those skilled in the art, especially in light of the detailed disclosure and examples provided herein.

The following non-limiting examples set forth below illustrate certain aspects of the invention.

### Examples

Materials and Methods

Materials—Wortmannin, nocodazole, cycloheximide, cyclodextrin were purchased from Sigma Aldrich. PD98059, LY294002 reagents were from EMD Biosciences. 8-pMeOPT-2-O-Me-cAMP (8-pMeOPT) and N6-Monobutyryladenosine-3′,5′-cyclic monophosphate (6-MB-cAMP) were from Axxora. BD adeno-X expression system, BD-Adeno-X virus purification kit, rapid titer kit were from Clontech (Mountain View, Calif.). MEM, FBS, trypsin-EDTA, Lipofectamine 2000, Dulbecco's PBS, penicillin-streptomycin were from Invitrogen. Antibodies for phospho-glycogen synthetic kinase 3-β (GSK3β), GSK3β, phospho-Akt and Akt were purchased from Cell Signaling. Anti-Epac1, Epac2, Rap1, anti-N-deacetylase/N-sulfotransferase-1 (NDST1) antibodies were purchased from Santa Cruz. Anti-syndecan-1 antibody was purchased from Invitrogen. Anti-anti-alpha-tubulin antibody was purchased from Abcam. Anti-HS antibody was purchased from Kamiya Biomedical.

Cell Culture—SK-Mel-2 and SK-Mel-24 (ATCC) cell lines were cultured in Eagle's Minimum Essential Medium supplemented with 10% fetal bovine serum at 37° C./5% CO₂. HEMA-LP human melanocytes (Cascade Biologics) were maintained in Medium 254 with Human Melanocyte Growth Supplement (Cascade Biologics).

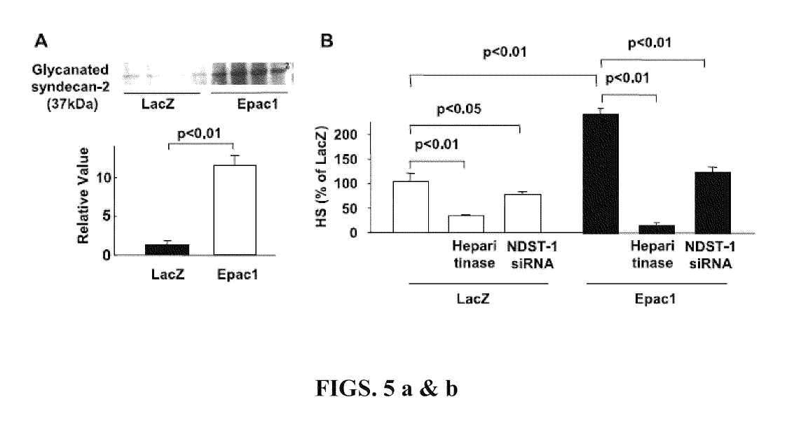

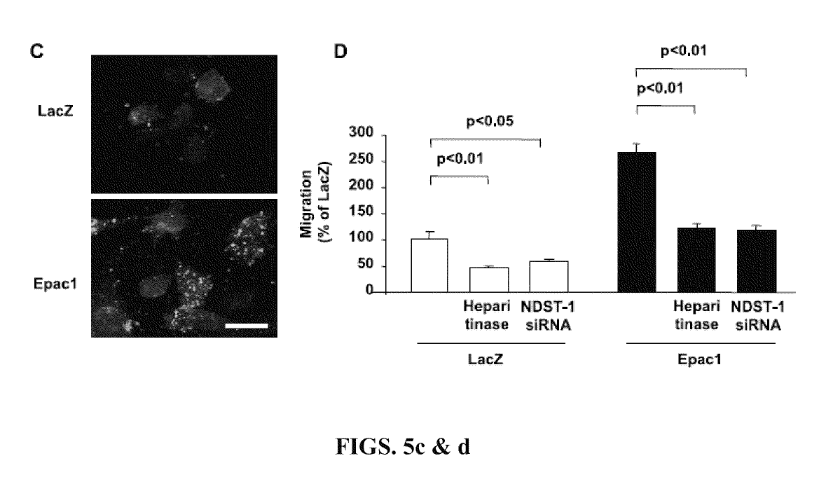

Adenoviral Overexpression—Recombinant adenoviruses containing human LacZ, Epac1 or Epac2 were constructed (Adeno-X Expression System, Clontech). Human Epac1 and Epac2 cDNA were kindly provided by Dr. J. L Bos (University Medical Center, Utrecht, Netherlands). Adenovirus of PKA alpha subunit was purchased from Vector Biolabs. The corresponding encoding sequence was cloned in topShuttle2 (Clontech) to obtain a mammalian expression cassette, which was then excised and ligated into BD Adeno-X Viral DNA. The recombinant vector was introduced into human embryonic kidney cells (HEK293) to recover infectious adenovirus. Viruses were propagated in HEK293 cells, and purified by BD Adeno-X Virus Purification Kits. Viral titer was determined by Adeno-X Rapid Titer Kit (Clontech). As a control study, adenovirus vector harboring LacZ was used at the same MOI. Cells were infected with adenovirus for 24 hand subjected to each experiment. In some experiments (For HS production and NDST-1 expression, FIGS. 5B, 5C, 5D, 5E, 5F and 5G), cells were further incubated for 48 h in medium without adenovirus followed by each experiment.

Quantitative Real Time PCR (qPCR)—qPCR was performed using methods previously known in the art. Total RNA was extracted using RNAeasy kit (QIAGEN), and then first-strand cDNA was synthesized using the Taqman RT reagents (Applied Biosystems). Real-time PCR was then carried out on a DNA Engine Opticon 2 system (MJ Research Inc.) using the SYBR Green qPCR kit (BioRad). The three sets of pre-designed primer mixes for each gene of interest were optimized. Following specific oligonucleotide primers mixes were used in this study. Epac1 (Hs_RAPGEF3_{—}1_SG (QT00003381, QIAGEN), Epac2 (Hs00199754-m1RAPGEF4, ABI), and NDST-1(Hs_NDST1_{—}1SG Quantitect Primer Assay(QT01002638, QIAGEN).

Migration assay—Migration assay was performed using methods previously known in the art using the Boyden chambers (pore size 8 μm, BD Biosciences). The upper chamber's polycarbonate insert film parts were coated by 75 μl fibronectin (50 μg/ml in PBS, Biosciences). Cultured cells were detached and the number of the cells was adjusted to 1×10³ cells/μl of media. One-hundred micro liter of the cell suspension was applied to the center of the upper chamber and then attached to the lower chamber. Thereafter, the cells were incubated in CO₂ incubator at 37° C. for 3 h unless specified. After fixation with 10% formalin neutral solution, cells were stained with Diff-Quick kit (Dade Behring). After mechanical removing of the cells on the upper surface of the membrane with a cotton swab, cells that migrated onto the lower surface of the membrane were counted. Pictures were taken with a microscope followed by counting migrated cells with Image J software in randomly chosen 10 fields.

Time-lapse videomicroscopy—Analysis of cell motility using time-lapse videomicroscopy was performed using methods previously known in the art. SK-Mel-2 cells overexpressing either LacZ or Epac1 were subjected to time-lapse video recording. Frames from the recording were digitized, and cell locations were identified at 30-minute intervals using either the centroids or nuclei. The speed of the cells was determined for distances between their successive positions.

Western Blotting—Western blot analysis was performed using methods previously known in the art. Cells were lysed and sonicated in lysis buffer containing 25 mM Tris-HCl (pH7.5), 150 mM NaCl, 5 mM MgCl₂, 1% NP-40, 1 mM DTT, 5% glycerol, phosphatase inhibitor (Sigma), protease inhibitor cocktail (Sigma) and 1 mM NaF. Equal amounts of protein (20 μg) were subjected to SDS-PAGE. After protein separation by electrophoresis, samples were transferred to Millipore Immobilon-P membrane and immunoblotting with antibodies was performed. Signal intensities of the bands were quantified with Image J software (NIH).

Immunoprecipitation—Immunoprecipitaiton was performed using methods previously known in the art. Cells were lysed in RIPA buffer containing 10 μg/ml aprotinin, 10 μg/ml leupeptin, 1 mM sodium orthovanadate and 1 mM phenylmethylsulfonyl fluoride. Immunoprecipitations were performed overnight at 4° C. using antibodies with protein A-Sepharose. Samples were then subjected to Western blot analysis. For syndecan-2 immunoprecipitation, beads for immunoprecipitation were subjected to heparitinase treatment for 4 h at 37° C. to separate syndecan-2 from HS chains.

Transfection of siRNA—Epac1 siRNA (Ambion), syndecan-2 siRNA (Ambion) and NDST-1 siRNA (QIAGEN) were transfected into subconfluent SK-Mel-2 cells using Lipofectamine 2000 (Invitrogen). For Epac1, a pool of double-stranded siRNAs containing equal parts of the following antisense sequence was used: 5′-AUCACUGUAUACCGGUUCC-3′ (SEQ ID NO: 1). For syndecan-2, a pool of double-stranded siRNAs containing equal parts of the following antisense sequence was used: 5′-CUCUGGACUCUCUACAUCC-3′ (SEQ ID NO: 2). For NDST-1, a pool of double-stranded siRNAs containing equal parts of the following antisense sequence was used: 5′-UUUAUUAGCAGUUAGUUCG-3′ (SEQ ID NO: 3). The corresponding non-targeting siRNA Silencer negative Control #2 siRNA (AM4613, Ambion) was used as a negative control. Twenty-four hours later, the medium containing siRNA was changed to fresh medium and incubated for 72 h. When siRNA transfection was combined with adenoviral infection, siRNA transfection followed the adenoviral infection.

HS ELISA—HS content was determined using methods previously known in the art with HS ELISA kit (Seikagaku). Cells were collected and disrupted by sonication followed by centrifugation at 14000 rpm for 10 min. The supernatants were collected and diluted 6 times. Twenty microliter of the diluted samples was incubated for 18 h at 4° C. in the plates coated with HS-antibody. The secondary reaction with HRP-conjugated streptavidin-biotinylated antibody was carried out for 1 h at RT. After color development and stop reaction, OD was measured at 350/630 nm.

Tubulin polymerization assay—Tubulin polymerization assay was performed using methods previously known in the art. SK-Mel-2 cells were washed gently with 2 ml prewarmed PBS twice. After adding 400 μl of microtubule-stabilizing buffer (MSB) containing 100 mM Tris/HCl (pH 6.75), 1 mM EGTA, 1 mM MgCl2, 2 M Glycerol, 0.1% Triton X100, 200 ρM phenylmethanesulfonyl fluoride (PMSF), 10 U/ml ETI and 20 μg/ml leupeptin, the cells were incubated for 15 min at 37° C. Then the cells were incubated again with 400 μl MSB containing 0.1% Triton-X for 15 min at 37° C. Eighty microliter of 72% TCA and 80 μl of 0.15% DOC were added to total 800 μl of the collected samples. The mixtures were incubated on ice for 10 min and centrifuged at 14000 rpm for 15 min. The pellets were resuspended with 100 μl of 100 mM NaOH and subjected to SDS-PAGE as a monomeric tubulin fraction The remaining cells were resuspended with 70 μl Lysis Buffer (50 mM Tris/HCl (pH 6.8), 1 mM EDTA, 1% SDS, 10% glycerol, 1 mM PMSF) and homogenated using a sonicator (1 sec, once) and subjected for SDS-PAGE as a polymeric tubulin fraction.

[³⁵S]Methionine pulse-labeling assay—[³⁵S]Methionine pulse-labeling assay was performed using methods previously known in the art. The cells were incubated with 100 μCi [³⁵S]Methionine for 18 h at 37° C. Then, the cells were lysed with immunoprecipitation buffer (0.5% Nonidet P-40, 0.5% deoxycholate, 0.1% SDS, 0.05M Tris-HCl, pH 7.5). Then, immunoprecipitation with anti-NDST-1 antibody was performed as described above. The amount of radiolabeled NDST-1 precipitate was analyzed by SDS-PAGE, and autoradiography overnight at 4° C.

Sucrose density gradient centrifugation—Lipid rafts-enriched membrane fractions were prepared using methods previously known in the art. Briefly, SK-Mel-2 cells were homogenated in 2 ml of 500 mM sodium carbonate (pH 11.0) with protease inhibitors (1 μg/ml leupeptin, 0.1 mM PMSF and 50 U/ml egg white trypsin inhibitor) and lysed by sonication. The lysate was then adjusted to 45% sucrose by mixing with 2 ml of 90% sucrose prepared in MBS buffer (25 mM 2-Morpholinoethanesulfonic acid (MES), pH 6.5, 0.15 mM NaCl) and placed at the bottom of 5% and 35% discontinuous sucrose gradient (in MBS buffer containing 100 mM sodium carbonate) for an overnight ultra-centrifugation (260,000 g). Fractions were removed sequentially from the top and designated as fractions 1 through 13. Then, fraction 6, lipid rafts-enriched fraction, was subjected to immunoprecipitation or western blot analysis. Flotillin, a lipid raft-associated protein, was used to confirm the existence of lipid rafts in this fraction.

Immunocytochemistry—Immunocytochemistry was performed using methods previously known in the art. SK-Mel-2 on glass coverslips were fixed, washed and permeabilized with 0.02% Triton-X followed by incubation with primary and secondary antibodies for 30 min at room temperature. Alexa Fluor 488- and 594-conjugated goat anti-rabbit or anti-mouse antibodies (Molecular Probes) were used. The pictures were taken with a digital camera operated on a Nikon Eclipse TE200 or a confocal microscope (Zeiss Axiovert 100M). For mounting media, Prolong-Gold antifade with DAPI (Molecular Probes) was used.

Lung colonization assay—To examine the metastatic potential of melanoma cells, lung colonization assay was performed using methods known in the art. In brief, Cloudman S91 melanoma cells (clone M3, European Collection of Cell Cultures) were maintained in Ham's F-10 (Sigma) with 2.5% FCS and 15% normal horse serum. The cells were infected with adenovirus expressing Epac1 or green fluorescent protein (GFP) and incubated for 36 h. The expression of Epac1 was examined by Western blot analysis. The cells were harvested and injected (2×10⁶ cells/0.2 ml) into tail veins of BALB/c nude mice (Charles River, Male, 6 weeks). Two weeks after the injection, the number of metastatic colonies on the surface of the lungs were counted under a dissection microscope. This study was approved by the Animal Care and Use Committee at Yokohama City University.

Statistical Analysis—All results are expressed as mean±SEM. Differences in all parameters between experimental groups were analyzed using Student's t-test or analysis of variances (ANOVA), followed by post-hoc analysis Fischer test for multiple observations. Differences were considered significant when p values were less than 0.05.

### Example 1

### Epac Increases Migration in Melanoma

The effect of target protein of cAMP, i.e, Epac and PKA, on cell migration in melanocyte (HEMA-LP) and melanoma cell lines (SK-Mel-2 and SK-Mel-24) was examined (FIG. 1A). 8-pMeOPT, an Epac-specific agonist, increased cell migration in melanoma cells lines, but not in a melanocyte cell line. By contrast, 6-MB-cAMP, a PKA agonist, did not increase cell migration in all cell lines. Adenoviral overexpression of Epac1 and Epac2, but not PKA, increased melanoma cell migration. Such overexpression of Epacs also increased melanocyte cell migration, suggesting that Epac provides migration ability.

In comparison between melanoma cell lines, basal migration was higher in SK-Mel-24 and SK-Mel-2 (FIG. 1B). When expression of Epacs was examined, mRNA expression of both Epac1 and Epac2 were higher in SK-Mel-24 than in SK-Mel-2 (FIG. 1C). In addition, Epac1 protein expression was also higher in SK-Mel-24 (FIG. 1D). These data implicated that the expression of Epac positively correlates with melanoma migration ability. In support, in SK-Mel-2, the combination of 8-pMeOPT and overexpression of Epacs further increased cell migration over the effect of Epac overexpression alone; however, such increase was not observed in SK-Mel-24 (FIG. 1A), indicating that cell migration was nearly saturated in SK-Mel-24 which has higher endogenous Epacs expression.

Since Epac1 expression was more abundant than Epac2 (FIG. 1C), the effect of deletion of Epac1 on melanoma cell migration was examined. When Epac1 expression was decreased by siRNA (FIG. 1E), migration was also decreased (FIG. 1F). In addition, the inhibitory effect of Epac1 siRNA was greater in SK-Mel-24 than in SK-Mel-2 (FIG. 1F) even though decrease of mRNA was similar between these cell lines (FIG. 1E). This is in accordance with the data that SK-Mel-24 showed higher Epac1 expression (FIGS. 1C and 1D) and increased basal migration (FIG. 1B) than SK-Mel-2. Video-recorded cell motility was also increased by Epac1 overexpression (FIG. 1G). Put together, these data suggested that Epac plays a major role in melanoma cell migration.

FIG. 1 supports the foregoing analysis and illustrates that Epac increases melanoma cell migration where

A) Epac increases melanoma cell migration. HEMA-LP, SK-Mel-2 and SK-Mel-24 were infected with adenovirus harboring LacZ, Epac1, Epac2, and PKA followed by the migration assay in the presence or absence of 50 μM 8-pMeOPT or 50 μM 6-MB-cAMP. Both 8-pMeOPT and Epac overexpression, but neither 6-MB-cAMP nor PKA overexpression, increased melanoma cell migration. *, p<0.01 vs LacZ. n=4.

B) Migration assay was performed in SK-Mel-2 and SK-Mel-24. Basal migration was higher in SK-Mel-24 than in SK-Mel-2. n=4.

C) mRNA expression of Epac1 and Epac2 mRNA is shown. qPCR demonstrated that both Epac1 and Epac2 mRNA expression were higher in SK-Mel-24 than in SK-Mel-2. *, p<0.01 vs Epac1. n=4.

D) Immunoblots for endogenous Epac1 in SK-Mel-24 and SK-Mel-2 is shown. Epac1 protein expression was higher in SK-Mel-24 than in SK-Mel-2. n=4.

E) Effects of Epac1 siRNA on the mRNA expression in SK-Mel-2 or SK-Mel-24 are shown. qPCR demonstrated that Epac1 siRNA decreased Epac1 mRNA in both SK-Mel-2 and SK-Mel-24. *, p<0.01 vs control siRNA. n=4.

F) Effects of Epac1 siRNA on basal cell migration in SK-Mel-2 or SK-Mel-24. Epac1 siRNA inhibited basal cell migration in both SK-Mel-2 and SK-Mel-24, and the degree of the inhibition was greater in SK-Mel-24 than in SK-Mel-2. *, p<0.01 vs control siRNA. n=4.

G) Analysis of cell motility of SK-Mel-2 using video-recording system is shown. SK-Mel-2 overexpressing Epac1 significantly increased cell motility. n=10.

### Example 2

### Epac-Induced Migration is Mediated by the Translocation of Syndecan-2

Changes in cell surface molecules which regulates cell migration were investigated. It was found that, in immunocytochemistry, Epac1 overexpression increased a particle size of syndecan-2 immunofluorescent signal (data not shown). Thus expression changes of syndecan-2 were examined; however, Epac1 overexpression did not increase syndecan-2 expression (data not shown). Since syndecan is known to translocate to lipid rafts, which serve as platforms for molecules involved in cell migration, it was hypothesized that such large particle indicates accumulation of syndecan-2 in lipid rafts. Immunocytochemistry showed that both Epac agonist and Epac1 overexpression increased co-localization of syndecan-2 with lipid rafts. CXD, a lipid rafts-disrupting agent, decreased Epac-induced syndecan-2 colocalization with lipid-rafts (FIG. 2A). Additionally, in lipid rafts-rich fraction purified from sucrose density gradient centrifugation, syndecan-2 expression was increased by Epac1 overexpression (FIG. 2B). These data suggested that Epac increased translocation of syndecan-2 to lipid rafts.

Next, whether translocation of syndecan-2 to rafts is involved in Epac-induced migration was examined. When lipid rafts was disrupted by CXD, Basal and Epac-induced migration was inhibited (FIG. 2C), suggesting that lipid rafts are necessary for Epac-induced migration. The effect of deletion of syndecan-2 on Epac-induced migration was then examined. When syndecan-2 expression was decreased with siRNA (FIG. 2D), Epac-induced migration was inhibited (FIG. 2C). These data suggested that syndecan-2 mediates Epac-induced migration, and implicated the translocation of syndecan-2 to lipid rafts is necessary for Epac-induced migration.

FIG. 2 supports the foregoing analysis and illustrates that Epac activates syndecan-2 translocation into rafts in SK-Mel-2 where

A) Immunocytochemical staining with syndecan-2 and FLAER is shown. (Top) Cells were incubated in the presence or absence of 50 μM 8-pMeOPT for 15 min. (Bottom) Cells were infected with LacZ or Epac1 adenovirus followed by incubation in the presence or absence of 10 μg/ml CXD. Both 8-pMeOPT and Epac1 overexpression increased colocalization of syndecan-2 with lipid rafts. CXD decreased Epac1-overexpression-induced colocalization of syndecan-2 with lipid rafts.

B) Immunoblot for syndecan-2 in lipid-rich fraction is shown. Cells with LacZ or Epac1 overexpression were subjected to sucrose density gradient centrifugation to purify lipid-rafts rich fraction. Lipid rafts were detected by flotillin. A bar graph shows densitometric analysis of syndecan-2 expression in the immunoblot above. Epac1 overexpression increased syndecan-2 expression in the rafts-rich fraction. *, p<0.01 vs LacZ, n=4.

C) Migration assay in cells with lipid rafts-disruption or deletion of syndecan-2 is shown. The migration assay was performed in cells overexpressing LacZ or Epac1 in the presence or absence of 10 μg/ml CXD. For LacZ-overexpressing cells, migration assay was performed for 8 h. In siRNA experiments, cells overexpressing Epac1 were transfected with control- or Epac1 siRNA followed by the migration assay. CXD and syndecan-2 siRNA decreased Epac1 overexpression-induced migration. n=4.

D) Immunoblot for syndecan-2 in cells transfected with control or syndecan-2 siRNA is shown. A bar graph shows densitometric analysis of the blot. n=4.

### Example 3

### Epac Translocates Syndecan-2 by Tubulin Polymerization

The mechanism by which Epac regulates syndecan-2 translocation was then examined. Since tubulin polymerization is known to mediate intracellular molecule transport, and syndecan-2 has a tubulin-binding motif in its intracellular domain, it was hypothesized that tubulin polymerization mediates Epac-induced translocation of syndecan-2. In melanoma cells, syndecan-2 indeed directly bound to tubulin (FIG. 3A). Such binding was not augmented by Epac1 overexpression, suggesting that Epac mediates syndecan-2 translocation not by enhancing the binding between syndecan-2 and tubulin. It was, thus, examined whether Epac increases tubulin polymerization, and whether it leads to syndecan-2 translocation. Epac1 overexpression increased polymer form of tubulin (FIG. 3B). In support, immunocytochemistry showed increased tubulin fine structure (FIG. 3C), which may reflect tubulin polymerization. These data suggested that Epac increases tubulin polymerization.

Next, whether inhibition of tubulin polymerization prevents syndecan-2 translocation to lipid rafts was examined. Immunocytochemistry showed that Nocodazole (NCD), a tubulin polymerization inhibitor, decreased the colocalization of syndecan-2 with lipid rafts (FIG. 3D). NCD also decreased expression of sydecan-2 in the lipid rafts-rich fraction (FIG. 3E). These data suggested that tubulin polymerization mediates Epac-induced syndecan-2 translocation to lipid rafts. Further, NCD inhibited Epac-induced migration (FIG. 3F), supporting the concept that tubulin polymerization mediates Epac-induced migration.

FIG. 3 supports the foregoing analysis and illustrates that Epac increases syndecan-2 translocation by modulating tubulin polymerization in SK-Mel-2 where

A) Immunoblots for tubulin and syndecan-2 in immunoprecipitation with syndecan-2 antibody are shown. Syndecan-2 physically bound to tubulin; however, Epac1 overexpression did not enhance the binding between syndecan-2 and tubulin.

B) Immunoblots for polymer and monomer form of tubulin are shown. A bargraph shows the densitometric analysis of ratios of tubulin polymers to tubulin monomers. Epac1 overexpression increased tubulin polymers. n=4.

C) Immunocytochemical staining with tubulin is shown. Cells overexpressing Epac1 were treated with NCD (10 μM) for 3 h. Epac1 overexpression increased fine tubulin network, and such network formation was inhibited by NCD. Scale bar, 3 μm.

D) Immunocytochemical staining with syndecan-2 and lipid rafts. Cells overexpressing Epac1 were treated with NCD (10 μM) for 3 h. NCD decreased Epac1-induced syndecan-2 colocalization with lipid rafts. Scale bar, 3 μm.

E) Immunoblot for syndecan-2 in lipid rafts-rich fraction is shown. Cells overexpressing Epac1 incubated with 10 μM NCD for 3 h followed by sucrose density gradient centrifugation. A bar graph shows densitometric analysis of syndecan-2 expression in the immunoblot above. NCD decreased syndecan-2 expression in lipid raft-rich fraction. n=4.

F) Migration assay was performed in cells overexpressing LacZ or Epac1 in the presence or absence of NCD (10 μM). NCD inhibited Epac1-induced migration. n=4.

### Example 4

### Epac Mediates Tubulin Polymerization via PI3 Kinase

The mechanism by which Epac regulates tubulin polymerization was then explored. Although a recent study demonstrated a direct binding of Epac to tubulin, this was not the case, at least, in melanoma cells; neither immunocytochemical studies nor immunoprecipitation assays showed association of Epac1 with tubulin in our study (data not shown). It is well known that the PI3 kinase regulates tubulin polymerization via the Akt/GSK3β pathway. Also, a report demonstrated that Epac activates PI3 kinase. Therefore, it was hypothesized that Epac increases tubulin polymerization via PI3 kinase. Wortmannin, a PI3 kinase inhibitor, inhibited Epac-induced tubulin polymerization (FIG. 4A). Wortmannin also inhibited Epac-induced phosphorylation of Akt (FIG. 4B) and GSK3β (FIG. 4C). These data implicated that Epac-induced tubulin polymerization is mediated presumably via the PI3K/Akt/GSK3β pathway. Wortmannin also decreased Epac-induced syndecan-2 localization in lipid rafts (FIGS. 4D and 4E), and migration (FIG. 4F), further supporting the involvement of PI3 kinase in Epac-induced migration.

FIG. 4 supports the foregoing analysis and illustrates that Epac regulates tubulin polymerization via PI3K in SK-Mel-2 where

A, B and C) Immunoblots for polymer and monomer form of tubulin (A), phosphorylated- and total-Akt (B) and phosphorylated- and total-GSK3β (C) are shown. Cells with Epac1 overexpression were incubated with 10 μM wortmannin for 3 h. A bargraph shows the densitometric analysis of ratios of tubulin polymers to tubulin monomers (A), and ratios of phosphorylated form and total protein (B and C). Akt to total Akt. Wortmannin decreased Epac1-induced tubulin polymerization, Akt and GSK3β phosphorylation. n=4.

D) Immunoblot for syndecan-2 in lipid rafts-rich fraction is shown. Cells overexpressing Epac1 were incubated with 10 μM wortmannin for 3 h followed by sucrose density gradient centrifugation. A bar graph shows densitometric analysis of syndecan-2 expression in the immunoblot above. Wortmannin decreased syndecan-2 expression in lipid raft-rich fraction. n=4.

E) Immunocytochemical staining with syndecan-2 and lipid rafts is shown. Cells overexpres sing Epac1 were treated with wortmannin (10 μM) for 3 h. Wortmannin decreased Epac1-induced syndecan-2 colocalization with lipid rafts. Scale bar, 3 μm.

F) Migration assay was performed in the presence or absence of wortmannin (10 μM). Wortmannin inhibited basal and Epac1-induced migration n=4.

### Example 5

### Epac Increases Melanoma Cell Migration via HS Production

Since lipid rafts serve as a platform for the binding of syndecans to the ECMs, the translocation of syndecan-2 is likely to augment the binding between melanoma cells and ECMs via syndecan-2. Because HS is a major component among syndecan-2-bound ECMs, whether translocation of syndecan-2 augments its binding to extracellular HS was examined. It was found that the glycanated form of syndecan-2, which reflects the HS-bound form of syndecan-2 (32), was increased by Epac1 overexpression (FIG. 5A). This data indicates that translocated syndecan-2 in lipid rafts augments its binding to extracellular HS, and in addition, also implicates that Epac increased the amount of extracellular HS itself. Thus, whether Epac increases HS production in melanoma cells was examined. Interestingly, Epac1 overexpression increased HS production as demonstrated by HS ELISA (FIG. 5B) and immunocytochemistry (FIG. 5C). These data suggested that Epac enhances melanoma cell migration not only by syndecan-2 translocation, but also by increased HS production. To investigate the involvement of HS production in Epac-induced migration, whether HS degradation inhibits migration was examined. When the amount of HS was decreased enzymatically with heparatinase (FIG. 5B), both basal and Epac-induced migration was decreased (FIG. 5D). This data was further confirmed by decreased Epac-induced migration with sodium chlorate, which chemically degrade HS (data now shown).

Next, the mechanism by which Epac increased HS production was explored. Changes in expressions of HS-biosynthetic enzymes was examined, and it was found that Epac1 overexpression markedly increased the expression of N-deacetylase/N-sulfotransferase-1 (NDST-1) (FIG. 5E). Also, it was found that Epac1 overexpression did not change NDST-1 mRNA expression nor protein degradation (data not shown), but, rather, at the level of proteins, as shown by increased translation in [³⁵S]methionine pulse-labeling assay (FIG. 5F). It was also examined whether deletion of NDST-1 decreases HS production and migration. When NDST-1 expression was reduced by siRNA (FIG. 5G), both basal and Epac-induced HS production was decreased (FIG. 5B), paralleled with decreased basal- and Epac-induced migration (FIG. 5D). Put together, these data suggested that Epac increases NDST-1 translation, which results in elevated HS production, and increased cell migration.

FIG. 5 supports the foregoing analysis and illustrates that Epac increases migration by HS production in SK-Mel-2 where

A) Immunoblot for glycanated form of syndecan-2 is shown. A bar graph shows densitometric analysis of the immunoblot above. Epac1 overexpression increased the glycanated form of syndecan-2. n=4.

B) Amount in intra- and extra-cellular HS is shown. Cells overexpressing Epac1 were incubated with heparitinase (0.08 U/ml) for 48 h. In NDST-1 siRNA, cells overexpressing Epac1 were transfected with NDST-1 siRNA after the termination of adenoviral infection, and incubated for 48 h. Epac1 overexpression increased HS. Haparatinase and NDST-1 siRNA decreased basal and Epac1-induced HS production. n=4.

C) Immunocytochemical staining with HS in cells overexpressing LacZ or Epac1 is shown. Scale bar, 3 μm.

D) Migration assay was performed with treatments of HS degradation or Epac1 deletion with siRNA. Cells overexpressing Epac1 were treated with heparitinase (0.08 U/ml) for 48 h after the termination of adenoviral infection. In NDST-1 siRNA, cells overexpressing Epac1 were transfected with NDST-1 siRNA after the termination of adenoviral infection, and incubated for 48 h. For LacZ-overexpressing cells, migration assay was performed for 8 h. Heparitinase and NDST-1 siRNA inhibited both basal and Epac1-induced migration. n=4.

E) Immunoblot for NDST-1 is shown. A bar graph shows densitometric analysis of the immunoblot above. Epac1 overexpression increased NDST-1 expression. n=4.

F) Autoradiography of NDST-1 in [³⁵S]-methionine pulse labeling assay is shown. Epac1 increased the amount of radio-labeled NDST-1. n=4.

G) mRNA expression of NDST-1 is shown. Cells were transfected with NDST-1 siRNA for 24 h followed by qPCR. n=4.

### Example 6

### Epac Increases Lung Metastasis of Melanoma, in Vivo

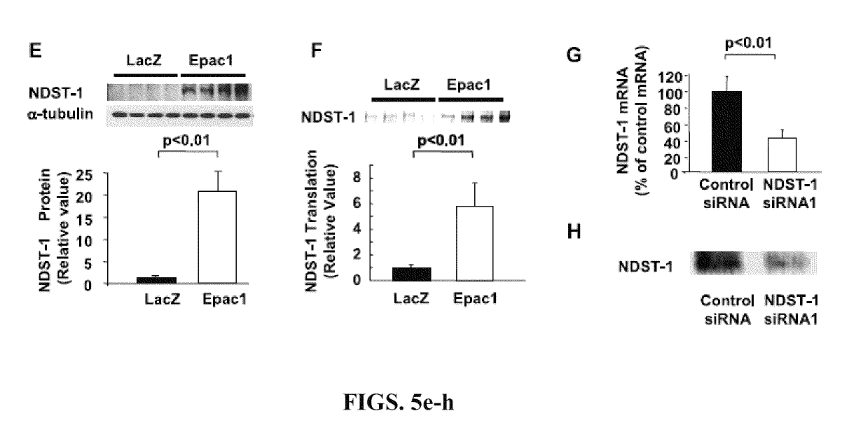

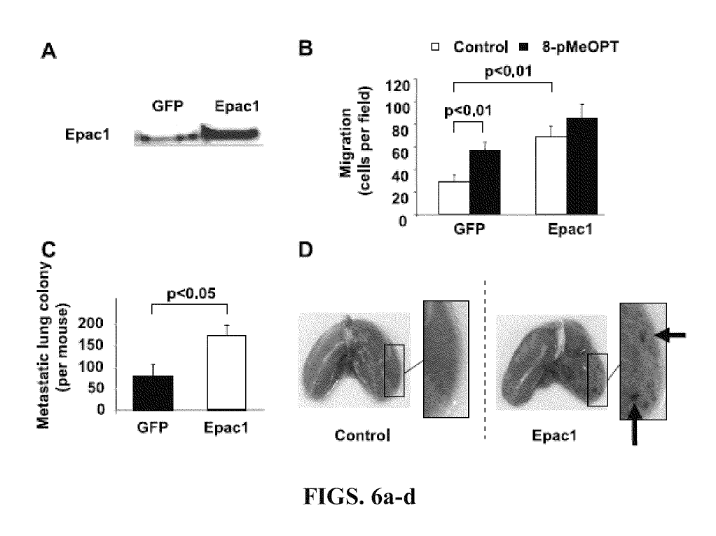

Since migration ability is essential for cancer metastasis, it was examined whether Epac increases melanoma metastasis by lung colonization assay in mice. In mouse Cloudman S91 melanoma cell line, Epac1 was endogenously expressed (FIG. 6A), and 8-pMeOPT increased migration (FIG. 6B). When Epac1 was overexpressed with adenovirus (FIG. 6A), migration was further increased (FIG. 6B). Thus, the effect of overexpression of Epac1 on melanoma metastasis was compared. The number of metastatic colonies in the lung was significantly higher in the Epac1-overexpression group than in GFP-overexpression (control) group (FIGS. 6C and 6D). Immunohistochemistry demonstrated that metastatic colonies in the Epac1-overexpression group showed increased Epac1 expression (FIG. 6E). This data suggested that Epac increases melanoma metastasis. Immunohistochemical study showed that expressions of HS and NDST-1 in the metastatic colonies were higher in Epac1-overexpression group than in GFP-overexpression group. These data implicated that Epac-induced melanoma metastasis is regulated presumably by HS-related mechanism.

FIG. 6 supports the foregoing analysis and illustrates that Epac increases lung metastasis of Cloudman S91 melanoma in mice.

A) Immunoblot for Epac1 is shown. Cloudman S91 cells express endogenous Epac1 (left lane). Adenoviral infection increased Epac1 expression (right lane).

B) Migration assay was performed in cells with GFP or Epac1 overexpression. Migration assay was performed in the presence or absence of 50 μM 8-pMeOPT. 8-pMeOPT and Epac1 overexpression increased migration. n=4.

C) The number of metastatic colonies in the lung of BALB/c athymic nude mice is shown. Cells with overexpression of GFP or Epac1 were injected from the tail vein. Two weeks after the injection, the number of metastatic colonies in the lung was counted. Epac1 overexpression increased the number of metastatic colonies. n=10.

D) Representative pictures of lungs 2 weeks after the melanoma cell injection are shown. Metastatic nodules on the lung surface are indicated by arrows.

E) Immunohistochemical staining for Epac1, HS and NDST-1 is shown. Expressions of Epac1 (top), HS (middle) and NDST-1 (bottom) were increased in metastatic colonies of the lung from mice which received injection of Epac1-orverexpressig cells. Nuclei were stained with DAPI (blue). Scale bar, 100 μm.

We claim:

1) A method for inhibiting, mitigating or preventing cancer metastasis in a subject diagnosed with a tumor expressing Epac comprising, administering an amount of one or more Epac inhibitors to said subject that is effective to inhibit tumor cell migration.

2) The method of claim 1 wherein the Epac inhibitor is a compound or a pharmaceutically acceptable salt thereof.

3) The method of claim 1 wherein the Epac inhibitor is a biological agent.

4) The method of claim 1 wherein the Epac inhibitor is a nucleic acid.

5) The method of claim 4 wherein the nucleic acid is selected from the group consisting of an encoding DNA enzyme, an antisense RNA, an siRNA, a shRNA, and an aptamer.

6) The method of claim 4 wherein the nucleic acid is an siRNA having an antisense sequence of SEQ ID NO: 1.

7) The method of claim 1 wherein the Epac inhibitor inhibits the expression of or activation of an Epac protein selected from the group consisting of Epac1 and Epac2.

8) A method for inhibiting, mitigating or preventing cancer metastasis in a subject diagnosed with a tumor expressing Epac comprising, administering an amount of one or more inhibitors to said subject, wherein the inhibitors target one or more proteins within an Epac-induced carcinoma migration pathway, and the amount of said inhibitors is effective to inhibit tumor cell migration.

9) The method of claim 7 wherein the inhibitor is a compound or a pharmaceutically acceptable salt thereof.

10) The method of claim 7 wherein the Epac inhibitor is a biological agent.

11) The method of claim 7 wherein the inhibitor is a nucleic acid.

12) The method of claim 11 wherein the nucleic acid is selected from the group consisting of an encoding DNA enzyme, an antisense RNA, an siRNA, a shRNA, and an aptamer.

13) The method of claim 11 wherein the nucleic acid is an siRNA inhibiting expression of syndecan-2.

14) The method of claim 13 wherein the nucleic acid is an siRNA inhibiting expression of syndecan-2 and having an antisense sequence of SEQ ID NO: 2.

15) The method of claim 11 wherein the nucleic acid is an siRNA inhibiting expression of NDST-1.

16) The method of claim 15 wherein the nucleic acid is an siRNA inhibiting expression of NDST-1 and having an antisense sequence of SEQ ID NO: 3.

17) The method of claim 7 wherein the inhibitor inhibits the expression of or activation of a protein selected from the group consisting of syndecan-2 and NDST-1.

18) A method for inhibiting, mitigating or preventing cancer metastasis in a subject diagnosed with a tumor expressing Epac comprising,
administering one or more Epac inhibitors to said subject in an amount effective to inhibit tumor cell migration; and
administering one or more second inhibitors to said subject in an amount effective to inhibit tumor cell migration, wherein the second inhibitors target one or more proteins within an Epac-induced carcinoma migration pathway.

19) The method of claim 1, wherein said tumor expressing Epac is a melanoma tumor.

20) The method of claim 8, wherein said tumor expressing Epac is a melanoma tumor.

21) The method of claim 18, wherein said tumor expressing Epac is a melanoma tumor.
